(19) United States
(12) Patent Application Publication
(10) Pub. No.: US 2006/0024392 A1
(43) Pub. Date: Feb. 2, 2006

(54) Compositions and methods for enhancing the effectiveness of a chemotherapeutic agent

(75) Inventors: Bernadene Ann Magnuson, Columbia, MD (US); Tao Yu, Laurel, MD (US)

Correspondence Address: ELMORE PATENT LAW GROUP, PC, 209 MAIN STREET, N. CHELMSFORD, MA, 01863, US

(73) Assignee: The University of Maryland, College Park, MD (US)

(21) Appl. No.: 11/106,240

(22) Filed: Apr. 14, 2005

Related U.S. Application Data
(60) Provisional application No. 60/563,778, filed on Apr. 20, 2004.

(51) Int. Cl. A61K 36/45

(52) U.S. Cl. 424/732

(57) ABSTRACT

This invention relates to food components such as phytochemicals including anthocyanins that can enhance the effectiveness of a chemotherapeutic agent in treating a cell proliferative disorder and/or reduce chemotherapy-induced side effects during treatment of a cell proliferative disorder.

### RELATED APPLICATIONS

This application claims the benefit of U.S. Provisional Application No. 60/563,778, filed on Apr. 20, 2004. The entire teachings of the above application are incorporated herein by reference.

### FIELD OF THE INVENTION

This invention relates to food components such as phytochemicals including anthocyanins that can enhance the effectiveness of a chemotherapeutic agent in treating a cell proliferative disorder and/or reduce chemotherapy-induced side effects during treatment of a cell proliferative disorder.

### BACKGROUND OF THE INVENTION

Colorectal cancer (CRC) is the second leading cause of cancer death in Western countries (American Cancer Society 2000). In recent years epidemiological studies have shown that consumption of a diet containing fruits and vegetables rich in antioxidants is linked to lower incidence of colon cancer (Yang et al., 2001, Annu Rev Nutr., 21:381-406). Many of the compounds in fruits and vegetables such as vitamins, minerals, phytochemicals are defined as dietary supplements (Dietary Supplement Health and Education Act, 1994). Among colorectal cancer patients it is reported that 64% use alternative medicine of which 57% take some kind of dietary supplement (Patterson et al., 2002, J. Altern Complement Med, 8:477-485). Various phytochemicals in fruits and vegetables have been shown to inhibit colon cancer development in animal model studies (Magnuson et al., 1998, American Chemical Society, 231-243), and in cell culture or in vitro studies (Malik et al., 2003, Nutr & Cancer, 46(2):186-196). But little attention has been paid to the potential of food extracts or phytochemicals to reduce chemotherapy-induced toxicity or adverse side effects in patients with pre-existing cancer.

Surgery, chemotherapy and radiation are the three most common forms of treatment for CRC. Chemotherapy uses one or more of several drugs to kill cancer cells, or to stop division and reproduction of cancer cells. Chemotherapy is used to kill any microscopic cancer that remains after surgery, control the spread or growth of tumor, and relieve cancer symptoms. The chemotherapy for CRC can be the primary treatment, or as adjuvant therapy with surgery and radiation treatments. Clinically, during the last 30 years, 5-fluorouracil (5FU) has remained the single most effective treatment for colon cancer despite a response rate of only 20% (Schmoll et al., 1999, Semin Oncol., 26:589-605; Tomiak, et al., 2000, Am J Clin., 23:94-98). 5FU is a fluorinated pyrimidine that is metabolized to its intracellular active form, fluorodeoxyuridine monophosphate (FdUMP), which inhibits DNA synthesis by inhibiting the normal production of thymidine. Some chemotherapeutic drugs and all radiation therapy generate free radicals and depend on them to damage the cancer cells. Theoretically, excess of free radicals can also lead to death of normal cells in the body when patients are treated with 5FU. Lipid peroxidation also leads to decrease of essential nutrients in the patients body and overall may be responsible for side effects including mouth sores and ulcers, diarrhea, gritty eyes and blurred vision, skin changes, suppression of bone marrow, nausea, vomiting, and hair loss. Side effects induced by 5FU are common and sometimes make patients unable to receive the next treatment cycle as scheduled (Tomiak, 2000, Am J Clin Oncol., 23:94-98).

There is great interest in identifying natural components that could reduce the side effects of chemotherapy. Lay literature and World Wide Web sites contain many claims that natural herbal treatments and extracts can reduce chemotherapy side effects, but to our knowledge there are no scientific studies to support these claims. Examples include Chinese herbs such as shen ling bai zhu tang (Ginseng, Poria and Atractylodes) and liu jun zi tang (Six Gentlemen Decoction), Qi-tonics and spleen-kidney herbal tonics (http://www.acupuncture.com), the herbs sundew (Drosera rotundifolia), periwinkle (Cathcranthus roseus), licorice (Glycyrrhiza glabra), and Essaic tea containing the four herbs burdock root (Arcticum lappa), Turkish rhubarb root (Rheum palmatum), sheep sorrel (Rumex acetosella), and slippery elm bark (Ulmus rubra) (http://www.consciouschoice.com/herbs/herbs1403.html). There are some natural dietary components that have been scientifically tested for their potential to enhance the activity of chemotherapeutic drugs. These include polyunsaturated fatty acids and various antioxidants (see review by Conklin, 2000, Nutr Canc., 37:1-18). For example, gamma linolenic acid from Borage has been reported to enhance effectiveness of lipophilic breast cancer chemotherapeutic drugs including tamoxifen (Kenny et al., 2000, Int J Cancer, 85:643-948) and vinorelbine (Menendez et al., 2002, Breast Cancer Res Treat, 72:203-219). Antioxidants that have been evaluated for their potential to enhance chemotherapeutic effectiveness include vitamin E, vitamin C, Coenzyme Q10, β-carotene, glutathione, glutamine, selenium, genistein and diadzein, and quercitin (Conklin, 2000, Nutr Canc., 37:1-18). The responsiveness depends on the cell type and chemotherapeutic agent.

The antioxidants, pyrrolidinedithiocarbamate and vitamin E reportedly enhanced the effectiveness of colon cancer treatment with 5FU (Chinery et al., 1997, Nat Med., 3:1233-1241). This effect was mediated by induction of p21WAF1/CIP1, a powerful inhibitor of the cell cycle, through a mechanism involving C/EBPbeta (a member of the CCAAT/enhancer binding protein family of transcription factors), independent of p53 (Chinery et al., 1997, Nat Med., 3:1233-1241). Similarly, curcumin has recently been reported to enhance the cytotoxicity of chemotherapeutic agents in prostate cancer by inducing p21 and C/EBP beta expression (Hour et al., 2002, Prostate, 51:211-218). Another postulated mechanism for the ability of antioxidants to enhance effectiveness of chemotherapeutic agents is to reduce the accumulation of lipid peroxidation products by cancer cells. Accumulation of lipid peroxidation products can reduce the rate of cancer cell proliferation causing them to be insensitive to the antineoplastic activity of the drug (Conklin, 2000, Nutr Canc., 37:1-18). However, conflicting reports have been reported and there is some concern that use of antioxidants during chemotherapy will reduce treatment efficacy (Conklin, 2000, Nutr Canc., 37:1-18).

There remains a need for identifying natural compounds, phytochemicals and food extracts that are effective to enhance the action of chemotherapeutic drugs and/or are effective to reduce side effects of chemotherapy.

### SUMMARY OF THE INVENTION

The present inventors have discovered that food extracts such as those described herein that are high in anthocyanins can enhance the effectiveness of chemotherapeutic agents and/or reduce chemotherapy-induced side effects during cancer treatment. The inventors have further discovered means for identifying food extracts and the components contained therein that are particularly effective to enhance the efficacy of chemotherapeutic agents and/or that are effective to reduce the undesirable side effects of chemotherapy.

### BRIEF DESCRIPTION OF THE DRAWINGS

FIG. 1 is a bar graph showing the effect of various concentrations of chokeberry anthocyanin-rich extract (ARE) in combination with the GI₅₀ concentration of 5FU, on cell growth of HT29 colon cancer cells.

FIG. 2 is a graph that shows enhanced growth inhibition of various concentrations of chokeberry ARE in combination with low doses of 5FU on HT29 colon cancer cells.

FIG. 3 is a graph showing the effect of combination of various concentrations of chokeberry ARE extract with low doses of 5FU on NCM460 normal colon cells.

FIG. 4 is a graph showing that growth inhibition of normal cells by 5FU is not enhanced by ARE.

FIG. 5 is a micrograph showing a comparison of the ileum (10×) of Group 2 mice treated with 5FU (Panel A) to the ileum (10×) of Group 4 mice treated with 5FU and chokeberry anthocyanin (Panel B).

### DETAILED DESCRIPTION OF THE INVENTION

In a first aspect of the invention, a method is provided to enhance the cytotoxic activity of a chemotherapeutic agent against a disorder of abnormal cell proliferation a patient comprising, administering an effective cytotoxicity-increasing amount of a composition comprising anthocyanins. In one embodiment of this aspect of the invention, the composition comprising anthocyanins is a berry extract rich in anthocyanins otherwise known as an anthocyanin-rich extract (ARE). In another embodiment of this aspect of the invention, the chemotherapeutic agent is 5 fluorouracil. In yet another embodiment of this first aspect of the invention, the disorder of abnormal cell proliferation in a patient is colon cancer.

In a second aspect of the invention, a method is provided to decrease the toxicity of a chemotherapeutic agent in normal cells of a patient undergoing chemotherapy comprising administering a composition comprising anthocyanins prior to, with, or following the chemotherapeutic agents. In one embodiment of this aspect of the invention, the composition comprising anthocyanins is a berry extract rich in anthocyanins. In another embodiment of this aspect of the invention, the chemotherapeutic agent is 5-fluorouracil. In yet another embodiment of the second aspect of the invention, the disorder of abnormal cell proliferation in a patient is colon cancer.

In a fifth aspect of the invention a method is provided to increase the therapeutic index of a chemotherapeutic agent administered for the treatment of abnormally proliferating cells comprising administering a composition comprising anthocyanins. In one embodiment of this aspect of the invention, the anthocyanins are derived from a food extract.

In a sixth aspect of the invention, a method is provided for identifying, in vitro, berry extracts effective in reducing chemotherapy induced toxicity by assessing the ability of the extract to decrease inhibition of cell growth on normal colon cells in the presence of the chemotherapeutic agents.

In a seventh aspect of the invention, a method is provided for identifying, in vitro, berry extracts effective in increasing the toxicity of a chemotherapeutic agent by assessing the ability of the extract to increase the growth inhibitory activity of in colorectal adenocarcinoma cells treated with chemotherapeutic agents.

In an eighth aspect of the invention, a method is provided for identifying, in vitro, phytochemicals effective in increasing the toxicity of a chemotherapeutic agent by assessing the ability of the phytochemical to increase growth activity in colorectal adenocarcinoma cells treated with a chemotherapeutic agent.

In a ninth aspect of the invention, a method is provided for identifying, in vitro, phytochemicals that increase the therapeutic index of a chemotherapeutic agent by assessing the effect of the phytochemical on the cytotoxicity (TD₅₀) and growth inhibitory (IC₅₀) activity of the chemotherapeutic agent in colon cancer cell lines and in normal colon cell lines.

In a tenth aspect of the invention, a natural berry extract rich in anthocyanins is provided as a phytoceutical/neutraceutical for enhancing the efficacy of a chemotherapeutic agent and/or for reducing the side effects of chemotherapy in a patient.

Anthocyanins are flavonoid pigments in blue and red fruits and vegetables. Bioflavonoids are the isoflavonoid and flavonoid compounds contained in certain foods such as berries. Phenolics are compounds with a phenyl group and having one or more hydroxyl groups contained in certain foods such as berries. Fruits and vegetables contain three main classes of dietary phenolics; flavonoids, phenolic acids and polyphenols. Over 5,000 different flavonoids have been described, and they are categorized into flavonols, flavones, catechins, flavanones, anthocyanins and isoflavonoids. In recent years a large number of investigators have studied how polyphenols and proanthocyanidins may act as anticancer agents by protection against free radical damage. The different hydroxylation, glycosylation and acylation patterns may modulate their antioxidative and biological activities. Acylated anthocyanins have been reported to have increased antioxidant activity as compared to their non-acylated counterparts (i.e. anthocyanidins) and studies suggest that antioxidant compounds may be effective against various cancers.

“Anthocyanin-rich extracts” or “AREs” are extracts derived from foods such as fruits and vegetables that are preferably, semi-purified, purified and/or concentrated such that the water content, sugar content and acid content are reduced and the remaining components are mainly the phenolics including anthocyanins. AREs are known in the art and many are readily available commercially from sources such as Artemis International, Inc. (Madera, Calif.). Concentrated and highly concentrated (about at least 2-3 grams of monomeric anthocyanin per liter or per kg) AREs obtained using standard separation and purification techniques and are also readily commercially available in the form of powders and liquids. Methods for obtaining AREs and isolated and concentrated anthocyanins are well known in the art (see, the Examples infra).

Fruits and vegetable that are particularly suitable for providing anthocyanin rich extracts include those fruits and vegetables preferably containing approximately at least 10 mg per 100 g of fresh fruit. In preferred methods and compositions of the invention, the AREs are derived and concentrated from berries and vegetables including but not limited to: chokeberry; raspberries of all kinds including black raspberry and red raspberry; blueberry; blackberry; cranberry; bilberry; black currant; cherry; elderberry; grape; kiwi; strawberry; purple potatoes; black carrots; or combinations of any of the above. Alternatively, blueberries and cherries extracts are not used. In one preferred embodiment, the ARE is derived from chokeberry, bilberry or grape or combinations thereof. In another preferred embodiment, the ARE is derived from chokeberry and the major anthocyanin contained in extract of chokeberry is cyanidin-3-galactoside.

The term “antineoplastic agents,” as used herein, refers to any substance that decreases abnormal cell proliferation. Antineoplastic agents have been described extensively in a number of texts, including Martindale, The Extra Pharmacopoeia, 31^{st} Edition, Royal Pharmaceutical Society (1996). A preferred antineoplastic for use in accordance with the invention is 5-fluoruracil.

Antineoplastic agents include: (i) antifolates; (ii) antimetabolites (including purine antimetabolites, cytarabine, fudarabine, floxuridine, 6-mercaptopurine, methotrexate, 5-fluoropyrimidine, including 5-fluorouracil, cytidine analogues such as β-L-1,3-dioxolanyl cytidine and 6-thioguanine); (iii) hydroxyurea; (iv) mitotic inhibitors (including CPT-11, Etoposide (VP-21)), taxol and vincristine; (v) alkylating agents (including but not limited to busulfan, chlorambucil, cyclophosphamide, ifofamide, mechlorethamine, melphalan and thiotepa); (vi) nonclassical alkylating agents, platinum containing compounds, bleomycin, anti-tumor antibiotics, anthracycline, anthracenedione, topoisomerase 11 inhibitors, hormonal agents (including but not limited to corticosteroids (dexamethasone, prednisone and methylprednisone); and (v) androgens such as fluoxymesterone and methyltestosterone, estrogens such as diethylstilbesterol, antiestrogens such as tamoxifen, LHRH analogues such as leuprolide, antiandrogens such as flutamide, aminoglutethimide, megestrol acetate, and medroxyprogesterone), asparaginase, carmustine, lomustine, hexamethyl-melamine, dacarbazine, mitotane, streptozocin, cisplatin, carboplatin, levamasole, and leucovorin.

Active compositions in accordance with the invention can be used to increase the cytotoxicity of antineoplastic agents to disorders of abnormal cellular proliferation, including, but not limited to: (i) benign tumors, including, but not limited to papilloma, adenoma, firoma, chondroma, osteoma, lipoma, hemangioma, lymphangioma, leiomyoma, rhabdomyoma, meningioma, neuroma, ganglioneuroma, nevus, pheochromocytoma, neurilemona, fibroadenoma, teratoma, hydatidiform mole, granuosa-theca, Brenner tumor, arrhenoblastoma, hilar cell tumor, sex cord mesenchyme, interstitial cell tumor and thyoma; (ii) malignant tumors (cancer), including but not limited to carcinoma, including renal cell carcinoma, prostatic adenocarcinoma, bladder carcinoma, and adenocarcinoma, fibrosarcoma, chondrosarcoma, osteosarcoma, liposarcoma, hemangiosarcoma, lymphangiosarcoma, leiomyosarcoma, rhabdomyosarcoma, myelocytic leukemia, erythroleukemia, multiple myeloma, glioma, meningeal sarcoma, thyoma, cystosarcoma phyllodes, nephroblastoma, teratoma choriocarcinoma, cutaneous T-cell lymphoma (CTCL), cutaneous tumors primary to the skin (for example, basal cell carcinoma, squamous cell carcinoma, melanoma, and Bowen's disease), breast and other tumors infiltrating the skin, Kaposi's sarcoma, and premalignant and malignant diseases of mucosal tissues, including oral, bladder, and rectal diseases, central nervous system tumors (glioblastomas), meningiomas, and astrocytomas; and (iii) hyperproliferative and preneoplastic lesions, including mycosis fungoides, psoriasis, dermatomyositis, rheumatoid arthritis, viruses (for example, warts, herpes simplex, and condyloma acuminata), molluscum contagiosum, remalignant and malignant diseases of the female genital tract (cervix, vagina, and vulva).

Of these, particular conditions that can be treated in accordance with the present invention includes colorectal cancer, ovarian cancer, bone cancer, renal cancer, breast cancer, gastric cancer, pancreatic cancer, melanoma, hematopoietic tumors such as lymphoma, leukemia, plasma cell dyscrasias, and multiple meyloma and amylodosis. In one embodiment, the invention is particularly suitable for the treatment of colon cancer.

A patient, including mammals, and specifically humans, suffering from any of the above-described conditions can be treated by the topical or systemic administration to the patient of an effective amount of an active composition in accordance with the invention in combination with an antineoplastic agent, preferably in the presence of a pharmaceutically acceptable carrier or diluent. By an “effective amount” of an active composition of the invention or chemotherapeutic agent of the invention is meant an amount which confers a therapeutic effect on the treated subject, at a reasonable benefit/risk ratio applicable to any medical treatment. The therapeutic effect may be objective (i.e., measurable by some test or marker) or subjective (i.e., subject gives an indication of or feels an effect). It will be understood, however, that the total daily usage of the compounds and compositions of the present invention will be decided by the attending physician within the scope of sound medical judgment. The specific therapeutically effective dose level for any particular patient will depend upon a variety of factors including the disorder being treated and the severity of the disorder; the activity of the specific compound employed; the specific composition employed; the age, body weight, general health, sex and diet of the patient; the time of administration, route of administration, and rate of excretion of the specific compound employed; the duration of the treatment; drugs used in combination or contemporaneously with the specific compound employed; and like factors well known in the medical arts.

The active composition in accordance with the invention can be administered prior to, in combination with, or following treatment with an antineoplastic agent. Methods and dosages for the administration of antineoplastic agents are known to those skilled in the art, and are described in a number of texts, including The Physician's Desk Reference, Martindale's The Extra Pharmacopeia, and Goodman & Gilman's The Pharmacological Basis of Therapeutics, or can be easily determined using standard methods.

In one embodiment, active compositions such as berry extracts and other food extracts rich in antioxidants and/or anthocyanins and other phytochemicals can be formulated for administration as a food supplement using one or more fillers. Neutraceutical compositions can be formulated for administration by any route including, but not limited to, inhalation, insufflation (through mouth or nose) oral, buccal, parenteral, vaginal or rectal administration. In one embodiment for oral administration, the active compositions are added directly to foods ingested as part of a normal meal. In this embodiment, the extracts may be in the form of dry powders or sachets for sprinkling directly on food or drink prior to ingestion. Various methods are known to those skilled in the art for addition or incorporation of nutraceuticals into foods. Alternatively, active compositions in accordance with the invention can be administered as conventional pharmaceuticals as is described below.

The active composition in accordance with the invention can be administered subcutaneously, intravenously, intraperitoneally, intramuscularly, parenterally, orally, submucosally, by inhalation, transdermally via a slow release patch, or topically, in an effective dosage range to treat the target condition. Typical systemic dosages for all of the herein described conditions are those ranging from 0.01 mg/kg to 500 mg/kg of body weight per day as a single daily dose or divided daily doses. Typical daily dosages can range from 0.1 mg-300 mg, preferably 1-200 mg, and more preferably between 60-100 mg. Typical dosages for topical application are those ranging from 0.001 to 100% by weight of the active composition. The compound is administered for a sufficient time period to alleviate the undesired symptoms and the clinical signs associated with the condition being treated. The active composition is included in the pharmaceutically acceptable carrier or diluent in an amount sufficient to deliver to a patient a therapeutic amount of active composition in vivo in the absence of serious toxic effects.

The concentration of active compound in the drug composition will depend on absorption, inactivation and excretion rates of the drug as well as other factors known to those of skill in the art. It is to be noted that dosage values will also vary with the severity of the condition to be alleviated. It is to be further understood that for any particular subject, specific dosage regimens should be adjusted over time according to the individual need and the professional judgment of the person administering or supervising the administration of the compositions, and that the dosage ranges set forth herein are exemplary only and are not intended to limit the scope or practice of the claimed composition. The active ingredient may be administered at once, or may be divided into a number of smaller doses to be administered at varying intervals of time.

A preferred mode of administration of the active compound for systemic delivery is oral. Oral compositions will generally include an inert diluent or an edible carrier. They may be enclosed in gelatin capsules or compressed into tablets. For the purpose of oral therapeutic administration, the active compound, can be incorporated with excipients and used in the form of tablets, troches or capsules. Pharmaceutically compatible binding agents, and/or adjuvant materials can be included as part of the composition.

The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

When the dosage unit form is a capsule, it can contain, in addition to material of the above type, a liquid carrier such as a fatty oil. In addition, dosage unit forms can contain various other materials which modify the physical form of the dosage unit, for example, coatings of sugar, shellac, or other enteric agents.

The compound or its salts can be administered as a component of an elixir, suspension, syrup, wafer, lozenge, chewing gum or the like. A syrup may contain, in addition to the active compounds, sucrose as a sweetening agent and certain preservatives, dyes and colorings and flavors.

The compound can also be mixed with other active materials that do not impair the desired action, or with materials that supplement the desired action, such as antibiotics, antifungals, antiinflammatories, antivirals, or other immunosuppressive agents.

Solutions or suspensions used for parenteral, intradermal, subcutaneous, or topical application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. pH can be adjusted with acids or bases, such as hydrochloric acid or sodium hydroxide. The parenteral preparation can be enclosed in ampoules, disposable syringes or multiple dose vials made of glass or plastic.

If administered intravenously, preferred carriers are physiological saline, bacteriostatic water, Cremophor EL.™. (BASF, Parsippany, N.J.) or phosphate buffered saline (PBS). In a preferred embodiment, the active compounds are prepared with carriers that will protect the compound against rapid elimination from the body, such as a controlled release formulation, including implants and microencapsulated delivery systems.

Suitable vehicles or carriers for topical application can be prepared by conventional techniques, such as lotions, suspensions, ointments, creams, gels, tinctures, sprays, powders, pastes, slow-release transdermal patches, suppositories for application to rectal, vaginal, nasal or oral mucosa. In addition to the other materials listed above for systemic administration, thickening agents, emollients and stabilizers can be used to prepare topical compositions. Examples of thickening agents include petrolatum, beeswax, xanthan gum, or polyethylene, humectants such as sorbitol, emollients such as mineral oil, lanolin and its derivatives, or squalene. A number of solutions and ointments are commercially available, especially for ophthalmic applications.

### EXAMPLES

### Example 1

In vitro screening methods to identify food extracts or phytochemicals that reduce chemotherapy-induced toxicity or side effects and to screen food extracts that have activity against colon cancer cell growth for their effect in combination with chemotherapy.

Methods:

1. Cell Lines:

HT-29, a cell line derived from colorectal adenocarcinoma (ATCC, HTB 38), grows in McCoy's 5A medium (Biowhittaker, Inc. Walkersville, Md.). NCM460, a cell line derived from a normal human colon (InCell Corp. San Antonio, Tex.), grows in M3 Base media (InCell Corp. San Antonio, Tex.).

Both cell lines are kept at 37° C. and 5% CO₂ atmosphere. Both media are supplemented with 10% fetal bovine serum (FBS, Biowhittaker, Inc. Walkersville, Md.).

2. Chemicals:

Sigma Chemical Company (St. Louis, Mo.).
Chokeberry Anthocyanin Extract (ARE):
   Standardized Chokeberry Powder (Artemis International, Inc.).
      S-Allyl-cysteine (SAC):

Tokyo Chemical Industry (Tokyo, Japan).

3. Sulforhodamine B (SRB) Assay:

Cells are fixed in situ by the gentle addition of 50 μl of cold 50% (w/v) TCA (final concentration, 10% TCA) and incubated for 60 minutes at 4° C. The supernatant is discarded, and the plates are washed five times with tap water and air dried. Sulforhodamine B (SRB) solution (100 μl) at 0.4% (w/v) in 1% acetic acid is added to each well, and plates are incubated for 10 minutes at room temperature. After staining, unbound dye is removed by washing five times with 1% acetic acid and the plates are air dried. Bound stain is subsequently solubilized with 100 μl 10 mM trizma base for 5 minutes, and the absorbance at wavelength of 490 nm was spectrophotometrically measured using a microplate reader Elx800 (Bio-Tek instruments, Inc.).

Experiment 1: Establish the GI₅₀ of 5FU in HT29 Colon Cancer Cells:

Experiments were conducted to establish the optimal methods for cell culture for determination of GI₅₀. Different densities of cells seeded (5,000 or 10,000 cells per well), fix methods and durations of the experiment were tried. The following method was determined to provide the most consistent and reproducible results (data not shown).

Cells were seeded in 96-well cluster tissue culture plates (Costar), containing 100 μl complete growth medium per well at densities of 5,000 cells per well. Incubated at 37° C. in a humidified tissue culture incubator. After 24 Hour, 100 μl complete medium containing 5 half-log dilutions of the 5FU from 2*10⁻⁴ to 2*10⁻⁶ M was added into each well (final concentrations are from 10⁻⁴ to 10⁻⁶ M). Six replicates were used for each concentration. Media containing 0.125% DMSO was used as control. After 48 hours of incubation with 5FU in medium, cell proliferation was assessed using the SRB assay.

Percentage growth (Growth%) is calculated as: [(Ti−Tz)/(C−Tz)]×100 [absorbance measurements at time zero (Tz), control growth (C), and test growth in the presence of drug at the different concentration levels (Ti)].

Growth inhibition of 50% (GI₅₀) is calculated from [(Ti−Tz)/(C−Tz)]×100=50, which is the drug concentration resulting in a 50% reduction in the net protein increase (as measured by SRB staining) in control cells during the drug incubation.

HT29 cells were exposed to serial dilutions of 5FU and the Growth percentage versus concentration curve was built for each experiment. GI₅₀ was calculated based on the curve. The experiment was repeated four times and the average of GI₅₀ (1.0E-4.37 M) was used for later experiments (Table 1). This value is very close to the GI₅₀ for 5FU reported by the National Cancer Institute, therefore we conclude the method we have establish is valid.

TABLE 1

| Conc. Of | 1 | | 2 | | 3 | | 4 | |
|---|---|---|---|---|---|---|---|---|
| 5FU | OD | Growth % | OD | Growth % | OD | Growth % | OD | Growth % |
| 1.0E−4 | 0.30 | 48.88 | 0.25 | 34.83 | 0.26 | 37.16 | 0.25 | 42.91 |
| 1.0E−4.5 | 0.32 | 54.63 | 0.27 | 44.48 | 0.27 | 43.58 | 0.27 | 53.85 |
| 1.0E−5 | 0.33 | 60.38 | 0.31 | 57.93 | 0.31 | 56.76 | 0.32 | 70.85 |
| 1.0E−5.5 | 0.40 | 82.11 | 0.37 | 77.24 | 0.37 | 76.69 | 0.36 | 87.85 |
| 1.0E−6 | 0.44 | 94.25 | 0.42 | 94.83 | 0.41 | 88.51 | 0.38 | 96.36 |
| Control | 0.46 | 100.00 | 0.44 | 100.00 | 0.44 | 100.00 | 0.39 | 100.00 |
| T0 | 0.15 | | 0.15 | | 0.15 | | 0.14 | |

| | | | | |
|---|---|---|---|---|
| GI50 | 1.0E−4.16 | 1.0E−4.61 | 1.0E−4.61 | 1.0E−4.28 |

| | |
|---|---|
| Ave GI50 | 1.0E−4.37 |

*control: 0.125% DMSO

Experiment 2: Determine the Effect of Combination of Various Concentrations of Chokeberry Extract with GI50 Concentration of 5FU on HT29 Growth:
1. Sample Preparation:

Chokeberry Anthocyanin-rich extract (ARE) was semi-purified by solid phase extraction using a C-18 cartridge (Waters Corp.). Color extract powder (1 g) was dissolved in 10 mL deionized water. Anthocyanins and other phenolics were bound to the cartridge while sugars and other polar compounds were washed away with 0.01% HCl acidified water. Anthocyanins and other phenolics were recovered with ethanol containing 0.01% HCl. The alcohol was then removed in a rotary evaporator at 40° C., and the solutes were re-dissolved in 10 ml of 0.01% HCl deionized water.

The monomeric anthocyanin content was determined by the pH-differential method. A Shimadzu 1601 UV spectrophotometer (Shimadzu Scientific Instruments, Inc., Columbia, Md.) and 1 cm path length disposable cells were used for spectral measurements at 520 and 700 nm. Pigment content was calculated as cyanidin-3-glucoside, using an extinction coefficient (ε) of 26,900 L cm-1 mol-1 and molecular weight (MW) of 449.2 g mol⁻¹.

2. Cell Growth Assay Experiment

Similar to the method used in testing GI₅₀ of 5FU: HT29 was plated at 5000 cells per well. After 24 hour, cells were exposed to 5FU at 1.0E-4.368 M (GI50 of 5FU in HT29 got from previous experiments) with chokeberry ARE at concentration of 1.56, 5, 15.6, 50 μg monomeric anthocyanin per milliliter growth media. After 48 hour treatment, cell growth was assessed using SRB assay. The data is shown in Table 2.

TABLE 2

| | OD | Growth % |
|---|---|---|
| Control | 0.39 | 100.00 |
| 5FU | 0.22 | 35.02 |
| 5FU + 1.56 ug/ml ARE | 0.22 | 33.85 |
| 5FU + 5 ug/ml ARE | 0.22 | 35.80 |
| 5FU + 15.6 ug/ml ARE | 0.21 | 28.79 |
| 5FU + 50 ug/ml ARE | 0.15 | 7.00 |

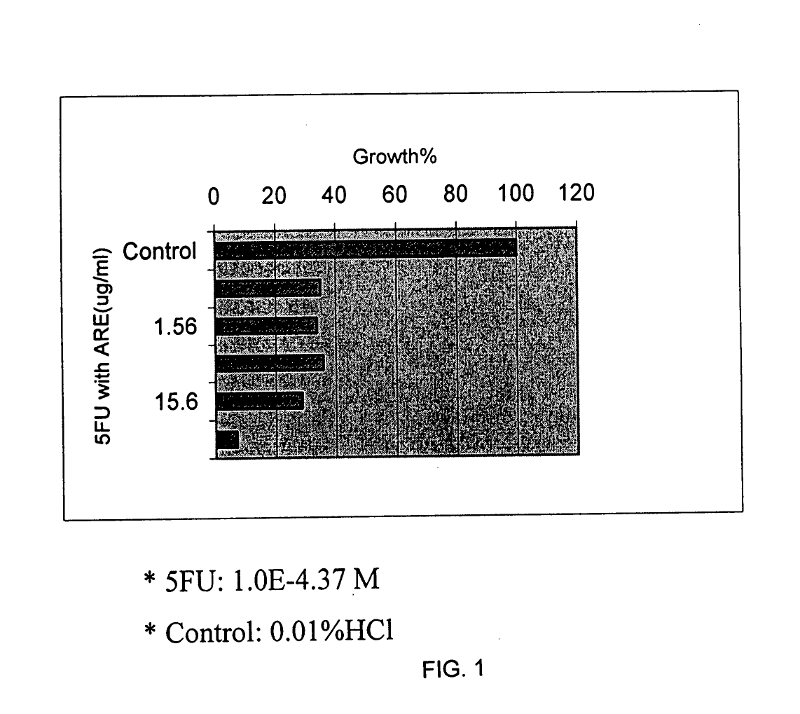

Using a concentration of 5FU equal to the GI50, low concentrations of chokeberry extract had little to no effect on HT29 cells (FIG. 1). The percentage of cell growth inhibition was very similar to cells treated with only 5FU and 0 extract. However, some additional growth inhibition occurred at 15.6 μg/ml, and this was significant when the concentration of chokeberry extract was 50 μg/ml. Compared to 5FU alone, which caused a 65% growth inhibition, 5FU plus 50 μg/ml chokeberry extract results in over 90% inhibition (FIG. 1).

Experiment 3: Determine the Effect of Combination of Various Concentrations of Chokeberry ARE Extract with Low Doses of 5FU on HT29 Colon Cancer Cells:

The protocol was the same as described above, except cells were exposed to 5FU at concentration ranging from 10⁻⁴ to 10⁻⁶ M with chokeberry ARE at concentration of 1.56, 5, 15.6, 50 μg monomeric anthocyanin/ml growth media. The data is shown in Table 3.

TABLE 3

| | ARE(μg/ml) | | | | | | | | | |
|---|---|---|---|---|---|---|---|---|---|---|
| Conc. of | 1.56 | | 5 | | 15.6 | | 50 | | 0 | |
| 5FU (M) | OD | Growth % | OD | Growth % | OD | Growth % | OD | Growth % | OD | Growth % |
| 1.0E−4 | 0.19 | 26.98 | 0.20 | 28.37 | 0.18 | 19.07 | 0.15 | 6.51 | 0.20 | 29.77 |
| 1.0E−5 | 0.25 | 53.49 | 0.25 | 52.56 | 0.21 | 34.42 | 0.17 | 14.42 | 0.24 | 49.30 |
| 1.0E−6 | 0.32 | 81.82 | 0.31 | 81.40 | 0.22 | 41.40 | 0.16 | 11.63 | 0.31 | 82.33 |
| 0 | 0.33 | 89.55 | 0.32 | 86.05 | 0.22 | 38.60 | 0.17 | 14.88 | 0.35 | 100.00 |

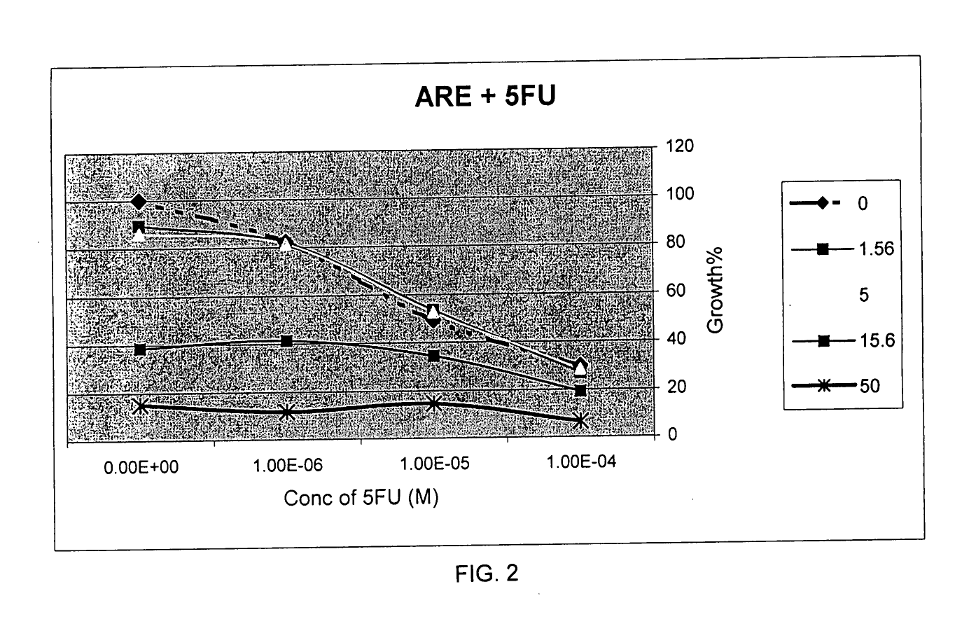

The significant inhibition of HT29 cells by ARE in the absence of 5FU is illustrated in the points corresponding to 0 concentration of 5FU, with HT29 cell growth (FIG. 2). At the lowest concentration of 5FU, the addition of 15.6 μg/ml of ARE greatly reduced the growth of cells from 80% of control down to only 40% of control (no treatment). At the intermediate concentration of 5FU, addition of 15.6 μg/ml of ARE reduced the growth of cells from 50% of control down to 35% of control cells. At the highest concentration of 5FU, addition of 15.6 μg/ml of ARE reduced the growth of cells from 30% of control down to only 20% of control cells.

Experiment 4: Establish the GI₅₀ of 5FU in NCM460 Normal Colon Cells:

Similar method as Experiment 1 was used, except cells were exposed to 5FU at concentration ranging from 10^{-4.5} to 10⁻⁷ M. Media containing 0.01% DMSO was used as control. Lower concentrations were used since it was observed that 0.1% DMSO inhibited the growth of NCM460 cells. The experiment was repeated 5 times. The data is shown in Table 4.

TABLE 4

| Conc. Of | 1 | | 2 | | 3 | | 4 | | 5 | |
|---|---|---|---|---|---|---|---|---|---|---|
| 5FU (M) | OD | Growth % | OD | Growth % | OD | Growth % | OD | Growth % | OD | Growth % |
| 1.0E−4.5 | 0.264 | 45.13 | 0.242 | 33.85 | 0.256 | 36.70 | | | | |
| 1.0E−5 | 0.281 | 53.85 | 0.27 | 48.21 | 0.275 | 45.41 | 0.285 | 57.58 | 0.279 | 51.41 |
| 1.0E−5.5 | 0.284 | 55.38 | 0.308 | 67.69 | 0.308 | 60.55 | 0.332 | 75.38 | 0.339 | 72.54 |
| 1.0E−6 | 0.286 | 56.41 | 0.299 | 63.08 | 0.32 | 66.06 | 0.38 | 93.56 | 0.381 | 87.32 |
| 1.0E−6.5 | 0.292 | 59.49 | 0.339 | 83.59 | 0.347 | 78.44 | 0.39 | 97.35 | 0.396 | 92.61 |
| 1.0E−7 | | | | | | | 0.396 | 99.62 | 0.388 | 89.79 |
| Control | 0.371 | 100.00 | 0.371 | 100.00 | 0.394 | 100.00 | 0.397 | 100.00 | 0.417 | 100.00 |
| T0 | 0.18 | | 0.18 | | 0.18 | | 0.13 | | 0.13 | |

| | | | | | |
|---|---|---|---|---|---|
| GI50 | 1.0E−4.85 | 1.0E−5.09 | 1.0E−5.14 | 1.0E−4.79 | 1.0E−4.93 |
| Ave GI50 | | | | | 1.0E−4.94 |

Experiment 5: Determine the Effect of Combination of Various Concentrations of Chokeberry ARE Extract with Low Doses of 5FU on NCM460 Normal Colon Cells:

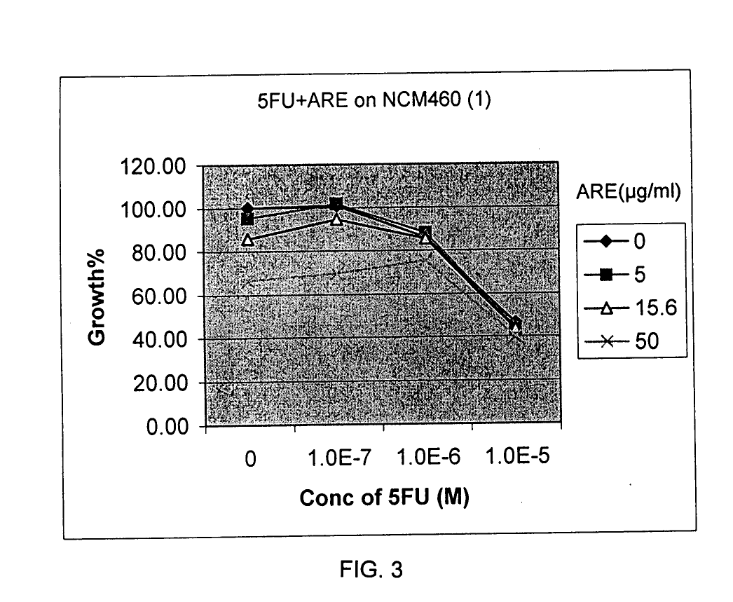

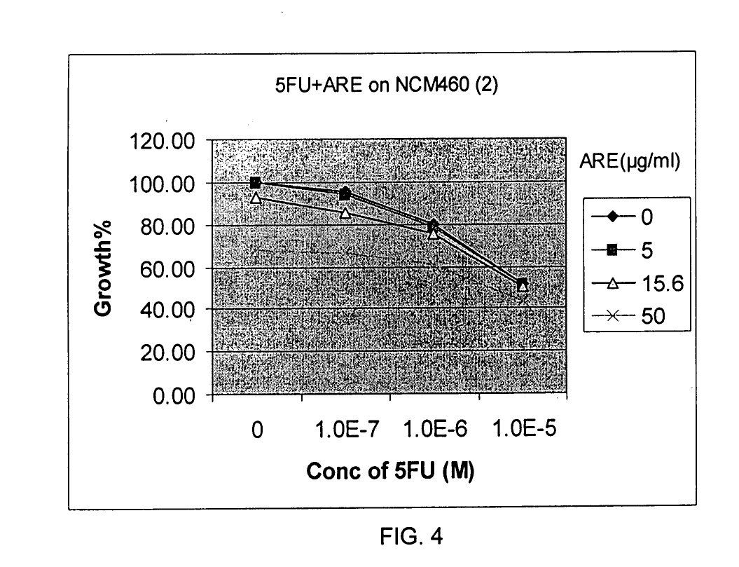

The protocol was the same as Experiment 3, except cells were exposed to 5FU at concentration ranging from 10⁻⁵ to 10⁻⁷ M with chokeberry ARE at concentration of 5, 15.6, 50 μg monomeric anthocyanin/ml growth media. FIGS. 3 and 4 show that growth inhibition of normal colon cells by 5 FU is not enhanced by ARE to the same degree as was observed in colon cancer cells (FIG. 2). Differences in response are most evident at the 5 FU concentration of 10⁻⁶ M and ARE concentration of 15.6 μg monomeric anthocyanin/ml. In normal cells, there is no difference in the percent growth by 5FU with or without ARE, whereas in colon cancer cells, growth with 5FU alone was 80% of control, and further reduced to 40% with ARE and 5 FU.

Experiment 6: Determine the Effect of Various Concentrations of SAC on Growth of HT29 Colon Cancer Cells:

SAC is a sulfur-containing compound from garlic which has been shown to have antioxidant activity (Yin et al., 2002, J. Agric. Food Chem., 50:6143-6147; Ho et al., 2001, Phytomedicine, 8(1):39-46). This experiment was designed to determine whether this antioxidant compound would have similar effects on HT20 as antioxidant anthocyanins.

The protocol was the similar to Experiment 1: Cells were exposed to half-log diluted SAC at concentration ranging from 3.2 to 1000 μM for 48 hour. Media containing 0.5% filtered water was used as control. The data is shown in Table 5.

TABLE 5

| | | |
|---|---|---|
| Conc. Of SAC | 1 | 2 |

| (μM) | OD | Growth % | OD | Growth % |
|---|---|---|---|---|
| 3.2 | 0.66 | 101.17 | 0.682 | 102.24 |
| 10 | 0.648 | 98.37 | 0.646 | 94.17 |
| 32 | 0.624 | 92.77 | 0.665 | 98.43 |
| 100 | 0.624 | 92.77 | 0.664 | 98.21 |
| 320 | 0.576 | 81.59 | 0.678 | 101.35 |
| 1000 | 0.599 | 86.95 | 0.666 | 98.65 |
| Control | 0.655 | 100.00 | 0.672 | 100.00 |

*control: 0.5% water

The data in Table 5 indicates that SAC did not show significant inhibition of HT29 cancer cells.
Experiment 7: Determine the Effect of Combination of Various Concentrations of SAC with Low Doses of 5FU on HT29 Colon Cancer Cells:

This experiment was to determine whether the antioxidant, SAC would enhance the effect of 5FU in HT29 cells. The protocol was the same as Experiment 3, except cells were exposed to 5FU at concentration ranging from 10⁻⁴ to 10⁻⁶ M with SAC at concentration of 10, 100, 1000 μM. The data is shown in Table 6.

TABLE 6

| | 5FU (M) | | | | | | | |
|---|---|---|---|---|---|---|---|---|
| | 0 | | 1.00E−06 | | 1.00E−05 | | 1.00E−04 | |
| SAC (uM) | OD | Growth % | OD | Growth % | OD | Growth % | OD | Growth % |

5FU + SAC (1)

| | | | | | | | | |
|---|---|---|---|---|---|---|---|---|
| 0 | 0.331 | 100.00 | 0.313 | 91.63 | 0.228 | 52.09 | 0.175 | 27.44 |
| 10 | 0.341 | 104.65 | 0.311 | 90.70 | 0.216 | 46.51 | 0.174 | 26.98 |
| 100 | 0.34 | 104.19 | 0.305 | 87.91 | 0.226 | 51.16 | 0.174 | 26.98 |
| 1000 | 0.321 | 95.35 | 0.283 | 77.67 | 0.22 | 48.37 | 0.168 | 24.19 |

5FU + SAC (2)

| | | | | | | | | |
|---|---|---|---|---|---|---|---|---|
| 0 | 0.353 | 100.00 | 0.304 | 79.32 | 0.219 | 43.46 | 0.178 | 26.16 |
| 10 | 0.335 | 92.41 | 0.298 | 76.79 | 0.22 | 43.88 | 0.177 | 25.74 |
| 100 | 0.333 | 91.56 | 0.31 | 81.86 | 0.216 | 42.19 | 0.182 | 27.85 |
| 1000 | 0.292 | 74.26 | 0.3 | 77.64 | 0.209 | 39.24 | 0.18 | 27.00 |

No significant enhancement of 5FU toxicity was observed with SAC, illustrating that the property of the ARE is unique and not simply as a result of antioxidant activity.
Conclusions:

1. The above data establishes a valid in vitro methodology for testing the effect of phytochemicals in combination with conventional chemotherapy for colon cancer.

2. The addition of chokeberry ARE to colon cancer cells enhanced the efficacy of growth inhibition by low dose 5FU, resulting in growth inhibition similar to the level observed with high doses of 5FU.

3. Natural fruits especially berries that are rich in antioxidants are promising agents to evaluate for enhancement of chemotherapy and reduction of side effects in colon cancer treatment. Total antioxidant activity may be important or the combination of various phytochemicals (anthocyanins and other phenolics) may be needed for the observed biological activity. Preliminary studies with blueberry extracts in our laboratory, which are reported to contain very high antioxidant activity, were not as effective as the chokeberry extract in inhibiting HT29 cell growth (data not shown). Similarly, SAC did not inhibit HT29 cell growth or enhance 5 FU toxicity to HT29 cells.

4. Testing extracts in other cancer cell lines with several chemotherapeutic drugs, natural components, will provide plant breeders with information on the components that may be bred in or out of the plant and elucidate mechanisms of action as to whether the interaction between the phytochemicals and 5FU are additive, antagonistic or synergistic.

### Example 2

In vitro and in vivo Studies to Screen for and Determine the Efficacies of Fresh Berry Extracts with Varying Amounts of Phytochemicals as having the Potential to Enhance 5 FU Cytotoxicity and/or reduce 5FU-Induced Toxicity or Adverse Effects

While the following experiment indicates that certain steps that will be taken in a particular order to complete the experiment, it is not meant to infer that all steps are necessary and/or that each step must be carried out in the particular order as they are described herein in order to successfully carry out the experiment as is readily ascertainable by one skilled in the art.

Research Design and Methods

Plant Material (Berries):

The Chief Breeder, University of Maryland, College Park, will provide all the varieties of fresh berries. The ten berries we will initially screen include: highbush blueberry (Vaccinium corymbosum), cranberry (Vaccinium macrocarpon), strawberry (Fragaria vesca), black currant (Ribes nigrum), blackberry (Rubus fruticosus), black raspberry, and four cultivars of red raspberry (Rubus idaeus). Either backcrosses or complimentary crosses between parent cultivars have genetically bred the new cultivars of raspberry. The four cultivars are:
(i) Anne (USPP#10412): a yellow raspberry that has very low levels of total phenolics and anthocyanins;
(ii) Caroline (USPP #10411): a red raspberry that has high levels of anthocyanins, ellagic acid, Se and fiber content;
(iii) Heritage: a purple raspberry with high content of total phenolics and anthocyanins;
(iv) QEG-f1: a red raspberry with different quality of anthocyanins from Caroline, its parent cultivar.

The selection of these berries was based on consultation with the breeder and reports of their total phenolic and anthocyanin content (Liu et al., 2002, J Agric Food Chem., 50:2926-2930).

Cell Lines:

NCM460, an epithelial cell line derived from normal colon cells (InCell Co. LLC), and HT-29 cells derived from colorectal adenocarcinoma (ATCC; HTB 38) will be used in the study. HT-29 grow as a monolayer in McCoy's 5A medium and NCM460 grow as monolayer/suspension mixture culture in M3:10 medium (InCell Co.), supplemented with 10% fetal bovine serum (FBS) and 1× antibiotic-antimycotic (Gibco-BRL) at 37° C. and 5% CO₂ atmosphere.

Animals:

Apc^{Min/+} mice in the C57BL/6J background and A/J mice will be purchased from the Jackson Laboratory (Bar Harbor, Me.). All rats will be single housed in plastic cages with wire tops under controlled conditions. Deionized water and food will be available at all times. Apc^{Min/+} mice are heterozygous for the Min (multiple intestinal neoplasia) allele, containing a nonsense mutation at codon 850, in the Apc (adenomatous polyposis coli) gene. Loss of the remaining wild-type Apc allele is one of the initial steps in adenoma formation. Familial adenomatous polyposis (FAP) is a human hereditary form of colorectal cancer that is also due to a germline mutation in Apc. In addition, somatic mutations in Apc are found in 80% of sporadic human colorectal cancer. The genetically inbred mice, A/J is highly susceptible to AOM and can develop tumors in distal colon as early as 6 weeks of AOM exposure (Papanikolaou et al, 2000, Carcinogenesis, 21:1567-1572).

Experimental Methods:

Berry Extract.

Two methods of berry extraction will be evaluated. The first will involve preparation of a freeze-dried whole berry extract as has been reported by others (Huang et al., 2002, Cancer Res., 62(23):6857-6863) and the second will be a simple extraction of the juice as would be easily accomplished by patients.

Whole Berry Extract

The whole berries will be freshly picked, washed and stored frozen at −20° C. within one day of picking. At time of analysis the berries will be freeze-dried in our tray freeze dryer, ground and extracted with acidified ethanol. Ground lyophilized material (500 mg) will be weighed and 10 ml of 80% acetone added, and the suspension stirred slightly. Tubes will be centrifuged (1500 g or 3000 g, 15 min), and the clear supernatant collected. The procedure will be repeated with another 10 ml of solvent and the supernatants combined and taken to dryness. The solid residue will be dissolved in acidified ethanol. Solid-phase extraction (SPE) using a C-18 cartridge (Waters Corp) (Malik et al., 2003, Nutr & Cancer, 46(2):186-196,) will be used to remove sugars and other polar compounds in the berry extracts in order to avoid interference in the antioxidant test and cell proliferation assay. Anthocyanins and other phenolics will be recovered with acidified ethanol. The alcohol will be removed in a rotary evaporator at 40° C., and the solutes redissolved in 1 ml of 0.01% HCl deionized water. To determine dry weights, a part of the extract will be lyophilized and the solid residue weighed.

Berry Juice Extract:

The whole berries will be freshly picked, washed and either juiced or stored frozen at −20° C. within 1 day of picking. Juice will be prepared using a consumer household juicer and either used immediately or frozen. At time of analysis the juice of the weighed berries will be extracted and passed through mira cloth (Fisher Scientific) to remove debris. The juice will passed through 0.2 micron filter before being lyophilized. A known amount of the powdered form of juice will be redissolved in 1 ml of 0.01% HCl deionized water or DMSO before cell proliferation assay. In our earlier work we have found that directly adding commercially available anthocyanin-rich extracts into cell growth media interfered with the visualization and analysis of the cell growth which was overcome by after the ARE went through SPE (unpublished). If it is found that the natural sugars found in the juice interferes with the cell proliferation assay then the reconstituted juice will be passed through C-18 cartridge before cell proliferation and antioxidant assays.

Cell Proliferation Assay:

We will use the 96-well growth plates and the Tox-6 kit (Sigma) to rapidly analyze the effects of different berry extracts on cell proliferation in our laboratory. This assay is protein based (Skehan et al., 1989, J Natl Cancer Inst., 82:1107-1112) and the cells are fixed without undergoing trypsinization. It was chosen to avoid the discrepancy by manual count when using Trypan blue and hemacytometer. The cells will be plated in 96-well plates (PGE Instruments) for 24 hrs before being exposed to different concentrations of the berry extracts and 5FU. Fresh medium, as is (Negative control A) and containing acidified water or DMSO (Negative control B) will be added to the cells. The changes in cell proliferation will be read spectrophotometrically and the cell number in each experiment will be related to a standard curve generated for the specific cell line. All experiments will be done in at least triplicate and will be repeated at least once. The amount of berry extract to be added to cells will be based on total phenolics/g fruit content. Initially, 5FU will be added at the IC50 concentration of 1×10⁻⁵ M. If inhibition of growth is observed then in addition to different concentrations, the effect of time of exposure will also be considered.
Experimental Design: 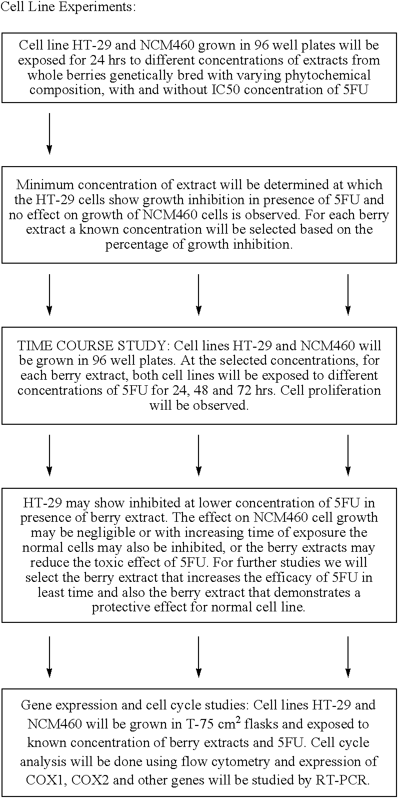
Animal Model Experiments:
Comparison of the Responsiveness of the Apc^{Min/+} and A/J Mice Models of Colon Cancer to 5FU

The fact that Apc^{Min/+} mice develop adenomas predominantly in the small intestine with few, if any, in the colon makes them an imperfect model for testing the effect of dietary compounds in human colorectal cancer as the environment and intestinal contents in the small intestine is very different than the colon. But at present it serves as the only mouse model available to study effect of 5FU on colorectal tumors. A/J mice are sensitive to carcinogen AOM that specifically forms tumors in colon. Tumors develop in the mice very quickly and can be observed within 2 months of carcinogen injection. In addition, these mice are much less expensive (about $20 each) compared to the Apc^{Min/+} mice. To our knowledge, there is no report of the response of chemically induced colonic tumors in A/J mice to 5FU. We will use this preliminary experiment to choose a model for evaluation of dietary treatments.

Animal Treatment:

To compare tolerance of and responsiveness of 5-FU in these 2 models, 5FU will be administered in three cycles of 40 mg/kg i.p. daily for 5 consecutive days followed by 9 days of rest for a total of 15 injections over a 5-week period as previously reported (Tucker et al., 2002, Cancer Letter, 187:153-162). Apc^{Min/+} mice and A/J mice (20 per group) will be fed standard rodent chow. For Apc^{Min/+} mice, 5-FU treatment will be started at 4 weeks of age, and animals will be killed at 10 weeks of age. Starting at 6 weeks of age, A/J mice will be injected intraperitoneally (i.p.) once a week for a total of 6 weeks with 10 mg/kg AOM according to the reported protocol (Papanikolsou et al, 2000, Carcinogenesis, 21:1567-1572). For A/J mice, 5-FU treatment will begin at 12 weeks of age (i.e. after the final dose of AOM.) and will continue for a 5-week period. A/J mice will be killed at 18 weeks of age.

Toxicity Evaluation:

Animal body weight, diarrhea, mouth ulceration, alopecia will be monitored daily. Gastrointestinal toxicity will be evaluated by characterizing the damage to the gastrointestinal tract as described by Tucker et al., 2002, Cancer Letter, 187:153-162. Briefly, sections of the small intestine and colon will be fixed in formalin. Tissues will be sent to a local contract histology laboratory (American Histology, Rockville, Md.) to be embedded in paraffin, sectioned and stained with hematoxylin and eosin. Sections will be examined for evidence of tissue damage, changes in crypt height and evidence of apoptotic bodies. The PI has experience in these techniques (Magnuson et al, 1994, Carcinogenesis, 15:1459-1462).

Evaluation of Response to 5FU:

For Apc^{Min/+} mice, the small and large intestines will be removed. For A/J mice, the entire colon will be removed. The intestines will be flushed with isotonic saline, slit open and fixed in formalin. The number, size and location of tumors will be recorded for all mice. Each tumor will be classified as being greater or less than 1 mm in diameter. After tumor scoring, the tumors will be dissected out, embedded in paraffin, sectioned and stained with hematoxylin and eosin. Tumors will then be examined and classified as either adenoma or adenocarcinoma. A significant reduction in the number of tumors per mouse, the size of tumors, or in the number of mice with tumors will be interpreted as evidence of response to 5FU. If the A/J mouse model is responsive to 5FU therapy, it will be used in the experiments to assess effect of combinational therapy with berry extracts. If it is not, the Apc^{Min/+} mice will be used.

Evaluation of a Combined Chokeberry Extract and 5FU Therapy in an Animal Model of Colon Cancer

We will test whether chokeberry extract either enhances the efficacy or reduces the toxicity of 5FU treatment in an animal model of colon cancer. We will use chokeberry as the test agent as we have demonstrated that this berry extract (1) significantly reduces cell proliferation specifically in colon cancer but not normal colon cell lines, (2) causes a block in the cell cycle, (3) reduces the number and size of preneoplastic colonic lesions in vivo, and (4) enhances the toxicity of 5FU to colon cancer cells in vitro. Therefore, although more potent extracts may be identified in Specific aim 1, we will use chokeberry for the initial experiment to test the hypothesis that extracts exhibiting these characteristics may be effective in vivo.

There will be 4 groups of 10 mice per group. All mice will be treated with 5FU. The 4 groups will be: (1) control fed the AIN-93G diet, (2) AIN-93G plus 10 mg/kg extract in diet, (3) AIN-93G plus 5 mg/kg extract in diet and (4) AIN-93G plus 1 mg/kg extract in diet. In our previous study with rats, 8 mg/kg was well tolerated and effectively reduced ACF formation. Therefore, we will test concentrations slightly higher and lower for efficacy with 5FU. Mice will be given the extract one week before and during 5FU treatment until the mice are terminated. Toxicity and evaluation of response will be conducted as described in 3.2.1.

Quality Control and Product Characterization:

There are many claims that natural herbs and extracts can reduce chemotherapy side effects. But the components and the purity of many herbs or mixture of extracts sold including herbs of the Fuzeng group, Qi-tonics, spleen-kidney herbal tonics etc. are not characterized. Many of the compounds in these plants are not stable over a long period of time and may loose their activity and byproducts may even be harmful for the cells. The berries that we propose to work with are natural food products, and the extracts we prepare will be characterized as described below, In addition the stability of each berry extract will be analyzed over time.

Total Phenolics and Anthocyanin Content:

Total soluble phenolics in the berries will be determined with Folin-Ciocalteu reagent according to the method of *Slinkard and Singleton (1977) using ellagic acid as a standard. Anthocyanin content will be determined by the pH-differential method using two different pH buffer solutions, 0.025M potassium chloride buffer (pH 1.0) and 0.4 M sodium acetate buffer (pH 4.5) as described previously (Malik et al., 2003, Nutr & Cancer, 46(2):186-196; Zhao et al., 2004, Journal of Food and Agricultural Chemistry, 52:6122-6128). A Shimadzu 1601 UV spectrophotometer (Shimadzu Scientific Instruments, Inc., Columbia, Md.) and 1 cm path length disposable cells will be used for spectral measurements at 520 and 700 nm. Pigment content will be calculated as cyanidin-3-glucoside, using an extinction coefficient of 26,900 L cm⁻¹ mol⁻¹ and molecular weight of 449.2 gmol⁻¹.

Measurement of Antioxidant Activity:

Oxygen Radical Absorbance Capacity (ORAC) Assay will be used to measure the antioxidant activity for berries following the method previously described by Cao et al., 1993, Cancer Letter Biol Med., 14:303-311. This assay measures the ability of antioxidant components in test materials to inhibit the decline in R-PE fluorescence that is induced by a peroxyl radical generator, AAPH. Phosphate buffer will be used as a blank and 1 μM Trolox (a water-soluble α-tocopherol analogue) as a standard during each run. The final volume of 2 ml is used in a 10 mm wide fluorometer cuvette. R-PE, phosphate buffer, and samples are preincubated at 37° C. for 15 min. The reaction will be started by the addition of AAPH. Fluorescence was measured and recorded every 5 min at the emission of 570 nm and excitation of 540 nm using a Shimadzu fluorometer until the fluorescence of the last reading declined to <5% of the first reading. One blank, one standard, and a maximum of 10 samples will be analyzed at the same time. The ORAC value refers to the net protection area under the quenching curve of R-PE in the presence of an antioxidant. The final results (ORAC value) are calculated and expressed using Trolox equivalents per gram of fresh weight (WM) or dry matter (DM) (Cao et al., 1993, Cancer Letter Biol Med., 14:303-311). Dry matter will be determined after lyophilization.
ORAC value (μM)=20K(Sₛₐₘₚₗₑ−S_{blank})/(S_{Trolox}−S_{blank})
K is a sample dilution factor, and S is the area under the fluorescence decay curve of the sample, Trolox or blank, which is calculated as
S=(0.5+f₅/f₀+f₁₀/f₀+f₁₅/f₀+f₂₀/f₀+f₂₅/f₀+f₃₀/f₀+ . . . +f₆₀/f₀+f₆₅/f₀+f₇₀/f₀)×5
where f₀ is the initial fluorescence at 0 min and fᵢ the fluorescence measurement at time i.
Mechanism of Action Analysis:
Cell Cycle:

To understand the mechanism by which these extracts act it is important to know the stage of the cell cycle that the treated cells are arrested in. Once the effect of extracts from berries is confirmed in 96-well plates, different cell lines will be plated in 75 cm² flasks (Falcon) and 24 hrs later berry extracts will be added at different concentrations. The cells will be trypsinized and collected after 24, 48 and 72 hrs time period. Light scattering characteristics and DNA content of the cells will be determined using a FACS calibur flow cytometer (Becton Dickinson, San Jose, Calif.). Washed cells will be fixed in ethanol and stained for DNA content. Flow cytometric datafiles will be collected for 10,000 events and analyzed using the CELLQuest program (Becton Dickinson). Cell cycle distribution percentages are calculated using ModFit LT software (Verity Software House, Inc., Topsham, Me.). Calibration standards (LinearFlow Green and DNA QC Particle Kit) for verification of instrument performance are purchased from Molecular Probes (Eugene, Oreg.) and Becton Dickinson, respectively. Dr. N. Schoene at Nutrition and Food Laboratory, USDA, Beltsville, Md. (Agreement letter attached) will be a consultant for flow cytometry and interpretation of these observations.

Gene Expression Analyses using RT-PCR:

RT-PCR will be used to study the changes in expression of cell cycle specific genes induced by various berries. We have already studied the changes in p21, p27 and other cell cycle related gene's expression in cells exposed to the chokeberry ARE (Malik et al., 2003, Nutr & Cancer, 46(2): 186-196). Changes in the gene expression were studied using gene specific primers in a multiplex PCR reaction, relative to a housekeeping gene. The semi-quantitative multiplex-PCR method uses 18S (housekeeping gene; 498 bp) as the internal standard. Changes in the expression of the gene of interest are represented by the changing ratio between the area of band representing the housekeeping gene and the gene specific band. PCR conditions for p21, p27, Cyclin A and Cyclin B are optimized (Malik et al., 2003, Nutr & Cancer, 46(2):186-196). DNA 500 LabChip and the Bioanalyzer 2100 (Agilent Technologies Inc. Rockville, Md.) will be used to analyze the changes in the gene expression. For gene expression study the cells will be grown in 25 cm² or 75 cm² flasks and the total RNA will be isolated using TRIzol (Invitrogen) and cDNA prepared by Retroscript kit (Ambion). Depending on the number of replicates most of the cells will be trypsinized, washed with 1× Phosphate Buffer and resuspended in RNALater (Ambion, Inc) to avoid degradation of RNA. Some of the other genes that will be analyzed include the cyclooxygenase genes, oncogenes and genes related to the MAPKinase pathway.

Statistical Analyses:

Multiple regression analyses of data from cell proliferation experiments, ORAC, total phenolics, total anthocyanins will be performed using the SAS System for Windows analysis package (Release 6.11, SAS) to assess the influence of these characteristics of the extracts on cell proliferation. All other data will be subjected to analysis of variance, and means were compared by least significant difference (LSD). The effect of species and cultivars on ORAC values and anthocyanin and total phenolic content will be evaluated by Fisher's LSD multiple-comparison test. Differences at p<0.05 were considered to be significant.

### Example 3

### Effect of Chokeberry Extract on 5FU Toxicity in Rats

The purpose of this experiment was to determine if the toxicity of 5FU would be decreased by feeding rats a diet supplemented with anthocyanin-rich extract (ARE) prior to and during 5FU treatment. Chokeberry ARE was used in this experiment as we have obtained the most detailed information on the mechanism of action of this extract (Malik et al, 2003, supra; Magnuson et al, (2005) Anthocyanin-rich extracts inhibit multiple biomarkers of colonic cancer in rats (submitted to Cancer Epidemiology, Biomarkers & Prevention).

The drug 5FU induces pathological changes in several organ systems, including the gastrointestinal tract, the kidney and the bone marrow. The damage to the gastrointestinal tract has been cited as the side-effect which most commonly causes patients to discontinue their course of treatment. Study design: Twenty-three male Sprague Dawley rats weighing 175-200 g were obtained from Charles River Laboratories. Rats were randomly allocated in two diet groups and maintained for two weeks on either AIN93 or AIN93 plus 4 g anthocyanin/kg (Table 7). This level of ARE inhibited early lesions of colon cancer in previous experiments (Malik et al, 2003, supra; Magnuson et al, (2005) Anthocyanin-rich extracts inhibit multiple biomarkers of colonic cancer in rats (submitted to Cancer Epidemiology, Biomarkers & Prevention). After 14 days on the diets, the following treatment groups were generated by treating five rats per group with either saline or 200 mg/kg 5FU.

TABLE 7

| Group/(Animal #)/total # | Diet | Treatment |
|---|---|---|
| 1/(1, 2, 3, 4, 5, 21, 24)/7 | AIN93 | Saline i.p. |
| 2/(6, 7, 8, 9, 10, 22)/6 | AIN93 | 5FU i.p. |
| 3/(11, 12, 13, 14, 15)/5 | AIN93 plus anthocyanin | Saline i.p. |
| 4/(16, 17, 18, 19, 20)/5 | AIN93 plus anthocyanin | 5FU i.p. |

The selected 5FU dose was reported in the literature to cause gastrointestinal damage, and a preliminary experiment with 2 rats illustrated that this dose caused weight loss and diarrhea, indicative of gastrointestinal damage.

Rats were given 1 g sucrose by oral gavage 1 hr after the 5FU or saline dose, then placed in a metabolic cage for 24 hr to collect urine. Urinary levels of sucrose are used as a measure of mucosal barrier integrity (Davies et al, 1995). Seventy-two hours after the 5FU injections, rats were euthanized by CO₂ inhalation. Blood was collected by heart puncture into heparinized tubes, the stomach and entire intestines were removed and stomach, duodenum, jejunum, ileum, cecum and colon sections were fixed in formalin for histopathological evaluation. In addition to the gastrointestinal tract, the following organs were also collected at necropsy for histopathological evaluation: heart, liver, kidney, spleen and bone marrow. These tissues were also fixed in formalin. All tissues were then routinely processed and stained with H&E for pathological evaluation.

Results:

Control tissues of Group 1 were within normal limits (WNL) with the exception of the liver of one animal (which presented with a small focal area of coagulation necrosis with suppurative inflammation).

Group 2 5FU treated animals on a regular diet showed the following toxic changes: panmyelophthisis of the bone marrow with congestion characterized by depletion of all cellular stages. The bone marrows of these animals were severely hypocellular affecting precursor and maturational stages of the erythroid and myeloid lineage alike. Remaining cells were megakaryocytes in significantly reduced numbers and few polymorphonuclear cells, most of which with eosinophilic granules. These effects are consistent with the known toxicity of 5FU to the bone marrow.

In the kidney, acute toxic nephropathy of the proximal convoluted tubules was present in all 5FU treated animals in mild to moderate severity. The lesion was characterized by degeneration and necrosis of tubular epithelium and formation of proteinaceous acellular casts in the tubular lumen. Interstitial renal edema and dilation of the urinary space were also observed.

In the small intestine, intestinal villi were moderately to severely denuded, dilation of lymphatic lacteals and crypt necrosis with crypt drop out affected the duodenum, the jejunum and the ileum equally severe. These changes are consistent with the known toxicity of 5FU to the gastrointestinal system.

Tissues of animals of group 3 treated with anthocyanin were overall similar to those of group 1. The following changes appeared more frequent in group 3 animals compared to controls: dilation of gastric glands, renal interstitial edema, bone marrow vacuolation and vacuolation of the small intestinal crypt and/or villi epithelium. All other changes were consistent with those sporadically observed in control animals.

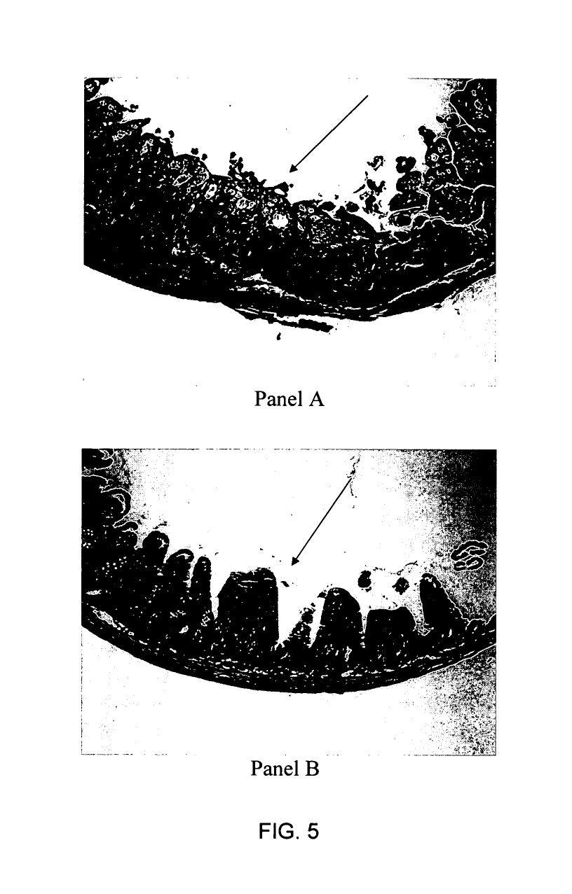

The animals of group 4, treated with 5FU and anthocyanin presented with lesions similar to those described for group 2. There was severe panmyelophthisis in the bone marrow, toxic nephropathy in the kidney, reduction of the mantel zone width in the spleen and denuded villi and crypt loss in the intestinal tract. The following observations were made: Bone marrow: The sections of group 4 contained generally more blood and vacuolation than group 2 tissues. The significance of this vacuolar change is uncertain. Kidney and spleen: the character and overall severity of the toxic nephropathy and the reduction of the mantel zone width in the spleen was similar among groups 2 and 4. Stomach: There appeared to be a greater incidence of glandular dilation and abscessation in the mucosal stomach of group 4- compared to group 2 animals. The significance of this change is uncertain. Small intestine: Although the character of the 5FU toxic change was similar in groups 2 and 4, the severity of the lesions appeared to be overall less in animals of group 4, which had also received anthocyanin in addition to the 5FU. The group 4 villi of the ileum were significantly less denuded as compared to the villi of the ileum of group 2 (see, FIG. 5). This finding suggests that fewer epithelial cells of the ileum were damaged by 5FU. It would be expected that reduced loss of absorptive epithelial cells of the villi would reduce loss of nutrients, reduce nausea and diarrhea associated with 5FU treatment. Large intestines: Changes observed appeared similar in character and severity among animals of groups 2 and 4.

Conclusion

The feeding of anthocyanin to normal animals did not result in a morphologically apparent effect with the exception of slight increases in bone marrow vacuolation, and vacuolation of the small intestinal epithelium. The significance of this vacuolation is uncertain.

Treatment with 5FU caused significant acute toxicological changes in the bone marrow, kidney, spleen, and gastrointestinal tract.

The feeding of anthocyanin preceding the 5FU treatment appeared to ameliorate the toxic effect of 5FU with respect to the severity of the gastrointestinal changes. This observation correlated with a proliferative blastic epithelial morphology of the crypt epithelium in anthocyanin treated animals, which were less severely affected by 5FU. Anthocyanin did not reduce bone marrow or renal toxicity of 5FU.

The patent and scientific literature referred to herein establishes the knowledge that is available to those with skill in the art. All United States patents and published or unpublished United States patent applications cited herein are incorporated by reference. All published foreign patents and patent applications cited herein are hereby incorporated by reference. All other published references, documents, manuscripts and scientific literature cited herein are hereby incorporated by reference.

While this invention has been particularly shown and described with references to preferred embodiments thereof, it will be understood by those skilled in the art that various changes in form and details may be made therein without departing from the scope of the invention encompassed by the appended claims.

What is claimed is:

1. A method to enhance the cytotoxic activity of a chemotherapeutic agent against a disorder of abnormal cell proliferation in a patient, comprising administering an effective amount of said chemotherapeutic agent in combination with an effective amount of a composition comprising anthocyanins.

2. A method to decrease the toxicity of a chemotherapeutic agent in normal cells of a patient undergoing chemotherapy comprising, administering a composition comprising anthocyanins prior to, with, or following the chemotherapeutic agent.

3. A method to increase the therapeutic index of a chemotherapeutic agent administered for the treatment of abnormally proliferating cells, comprising administering a composition comprising anthocyanins prior to, with, or following the chemotherapeutic agent.

4. The method of claim 1, wherein the abnormal cell proliferation is colorectal cancer.

5. The method of claim 1 wherein said chemotherapeutic agent is 5-fluorouracil.

6. A method to enhance the cytotoxic activity of a chemotherapeutic agent against a disorder of abnormal cell proliferation in a patient, comprising administering an effective amount of said chemotherapeutic agent in combination with an effective cytotoxicity-increasing amount of a berry extract rich in antioxidants.

7. A method to decrease the toxicity of a chemotherapeutic agent in normal cells of a patient undergoing chemotherapy comprising, administering a berry extract rich in antioxidants prior to, with, or following the chemotherapeutic agent.

8. A method to increase the therapeutic index of a chemotherapeutic agent administered for the treatment of abnormally proliferating cells, comprising administering a berry extract rich in antioxidants prior to, with, or following the chemotherapeutic agent.

9. The method of claim 6 wherein the abnormal cell proliferation is colorectal cancer.

10. The method of claim 7 wherein the abnormal cell proliferation is colorectal cancer.

11. The method of claim 8 wherein the abnormal cell proliferation is colorectal cancer.

12. The method of claim 6 wherein said chemotherapeutic agent is 5-fluorouracil.

13. The method of claim 7 wherein said chemotherapeutic agent is 5-fluorouracil.

14 The method of claim 8 wherein said chemotherapeutic agent is 5-fluorouracil.

15. The method of claim 6 wherein said berry extract is selected from the extract of chokeberry, raspberry, blueberry, blackberry, cranberry, bilberry, black currant, cherry, elderberry, grape, kiwi, strawberry or any combination thereof.

16. The method of claim 7 wherein said berry extract is selected from the extract of chokeberry, raspberry, blueberry, blackberry, cranberry, bilberry, black currant, cherry, elderberry, grape, kiwi, strawberry or any combination thereof.

17. The method of claim 8 wherein said berry extract is selected from the extract of chokeberry, raspberry, blueberry, blackberry, cranberry, bilberry, black currant, cherry, elderberry, grape, kiwi, strawberry or any combination thereof.

18. The method of claim 6 herein said berry extract is selected from the extract of chokeberry, raspberry, blackberry, cranberry, bilberry, black currant, elderberry, grape, kiwi, strawberry or any combination thereof.

19. The method of claim 7 wherein said berry extract is selected from the extract of chokeberry, raspberry, blackberry, cranberry, bilberry, black currant, elderberry, grape, kiwi, strawberry or any combination thereof.

20. The method of claim 8 wherein said berry extract is selected from the extract of chokeberry, raspberry, blackberry, cranberry, bilberry, black currant, elderberry, grape, kiwi, strawberry or any combination thereof.

21. The method of claim 6 herein said berry extract is a raspberry extract.

22 The method of claim 7 wherein said berry extract is a raspberry extract.

23. The method of claim 8 wherein said berry extract is a raspberry extract

24. The method of claim 6 wherein said berry extract is a chokeberry extract.

25. The method of claim 7 wherein said berry extract is a chokeberry extract

26. The method of claim 8 wherein said berry extract is a chokeberry extract

27. The method of claim 1 wherein the composition comprising anthocyanins comprises an extract selected from, chokeberry extract, raspberry extract, blueberry extract, blackberry extract, cranberry extract, bilberry extract, black currant extract, cherry extract, elderberry extract, grape extract, kiwi extract, strawberry extract, purple potato extract, black carrot extract or any combination thereof.

28. The method of claim 2 wherein the composition comprising anthocyanins comprises an extract selected from, chokeberry extract, raspberry extract, blueberry extract, blackberry extract, cranberry extract, bilberry extract, black currant extract, cherry extract, elderberry extract, grape extract, kiwi extract, strawberry extract, purple potato extract, black carrot extract or any combination thereof.

29. The method of claim 3 wherein the composition comprising anthocyanins comprises an extract selected from, chokeberry extract, raspberry extract, blueberry extract, blackberry extract, cranberry extract, bilberry extract, black currant extract, cherry extract, elderberry extract, grape extract, kiwi extract, strawberry extract, purple potato extract, black carrot extract or any combination thereof.

30. An in vitro method for identifying a berry extracts effective in reducing chemotherapy-induced toxicity by assessing the ability of the extract to decrease inhibition of cell growth on normal colon cells in the presence of the chemotherapeutic agent.

31. A in vitro method for identifying a berry extract effective in increasing the toxicity of a chemotherapeutic agent comprising, assessing the ability of the extract to increase growth inhibitory activity in colorectal adenocarcinoma cells treated with a chemotherapeutic agent.

32. An in vitro method for identifying phytochemicals effective in increasing the toxicity of a chemotherapeutic agent comprising, assessing the ability of the phytochemical to increase growth activity in colorectal adenocarcinoma cells treated with a chemotherapeutic agent.

33. An in vitro method for identifying phytochemicals that increase the therapeutic index of a chemotherapeutic agent comprising, assessing the effect of the phytochemical on the cytotoxicity (TD₅₀) and growth inhibitory (IC₅₀) activity of the chemotherapeutic agent in colon cancer cell lines and in normal colon cell lines.

34. A method to enhance the cytotoxic activity of 5-fluorouracil in the treatment of colon cancer in a patient, comprising administering to the patient an effective amount of 5 fluorouracil in combination with an effective cytotoxicity-increasing amount of a fruit or vegetable extract rich in anthocyanins.

35. A method to decrease the cytotoxicity of 5-flurouracil in the chemotherapeutic treatment of colon cancer in a patient comprising administering to the patient a therapeutically effective amount of a fruit or vegetable extract rich in anthocyanins.

36. The method of claim 34 wherein the fruit or vegetable extract is selected from chokeberry extract, raspberry extract, blueberry extract, blackberry extract, cranberry extract, bilberry extract, black currant extract, cherry extract, elderberry extract, grape extract, kiwi extract, strawberry extract, purple potato extract, black carrot extract or any combination thereof.

37. The method of claim 35 wherein the fruit or vegetable extract is selected from chokeberry extract, raspberry extract, blueberry extract, blackberry extract, cranberry extract, bilberry extract, black currant extract, cherry extract, elderberry extract, grape extract, kiwi extract, strawberry extract, purple potato extract, black carrot extract or any combination thereof.
